(19) United States
(12) Patent Application Publication
(10) Pub. No.: US 2025/0332425 A1
(43) Pub. Date: Oct. 30, 2025

(54) LEAD WIRE SLIDE, ASSEMBLY GROUP, AND MEDICAL SYSTEM

(71) Applicant: AMBU A/S, Ballerup (DK)

(72) Inventor: Line Leach KASTELIJN, København Ø (DK)

(73) Assignee: AMBU A/S, Ballerup (DK)

(21) Appl. No.: 19/190,533

(22) Filed: Apr. 25, 2025

(30) Foreign Application Priority Data
Apr. 26, 2024 (EP) 24172765.0

(51) Int. Cl. A61N 1/375; H01R 31/06; H01R 107/00

(52) U.S. Cl. CPC A61N 1/3752; H01R 31/06; H01R 2107/00; H01R 2201/12

(57) ABSTRACT

A lead wire slide (2), a medical system (38) comprising the lead wire slide (2), and an assembly group (34) comprising the first lead wire slide (2) and a second lead wire slide (2′). The lead wire slide (2), for slidable accommodation of medical lead wires (4), includes a base section (6) that includes a plurality of through-holes (8) that are each configured to accommodate one medical lead wire (4) in a slidable manner and a connecting section (10) that is provided on a side (12) of the base section (6) and configured to be connected to a complementary connecting section (10′) of another lead wire slide (2′).

### CROSS-REFERENCE TO RELATED APPLICATIONS

This application claims priority from and the benefit of European Patent Application No. 24172765.0, filed Apr. 26, 2024; the disclosure of said application is incorporated by reference herein in its entirety.

### TECHNICAL FIELD

The present disclosure relates to a lead wire slide for slidable accommodation of medical wires, an assembly group comprising lead wire slides, and a medical system comprising a lead wire slide.

### BACKGROUND

Electroencephalography (EEG) is a method or test to record an electrogram of the spontaneous electrical activity of the brain. Typically, EEG electrodes are placed along the head of a patient to measure voltage fluctuations, allowing to evaluate the brain activity and to diagnose brain diseases and brain disorders. The electrodes are often placed on the head using the so-called 10-20 system, wherein a minimum of 21 electrodes are used. The “10” and “20” refer to the fact that the actual distances between adjacent electrodes are either 10% or 20% of the total front-back or right-left distance of the skull. There already exist newer and finer systems, for example the 10-10 system or the 10-5 system, using even more electrodes with correspondingly less distance in between. Usually, each electrode is connected to a lead wire in an EEG device.

If a device, like an EEG device, has many lead wires, these may become curled up, tangled or twisted. For example, the lead wires may become tangled during transportation or handling. Such a lead wire bundle may then make a messy impression. It also makes it more difficult to find and grab a specific lead wire.

Lead wire slides may be made in one piece. Also, lead wires can be fixed and attached to an adapter, connector or clamp. However, such a rigid system prevents that individual lead wires are movable, for example if one lead wire is to reach a more distant location than another.

Existing lead wire slides are usually made of at least two connectable pieces, wherein the lead wires are placed in between. The two pieces can be understood as halves, that usually both have grooves, and when the halves are connected, one groove of one half forms together with a groove of the other half a hole through which a lead wire can run. The lead wires are inserted into a first half and then the second half is mounted on top of the first half.

Specifically for medical applications with electrodes attached to the lead wires, it is important that the lead wires are organized. That makes it easier to attach the electrodes to a patient. It should also be prevented that when one lead wire is pulled, for example to attach its electrode to the patient, another lead wire is also moved and then, for example, an electrode already attached to a patient is detached in the process. As mentioned above, many lead wires with electrodes placed closely together are used in electroencephalography (EEG) in particular, making it especially necessary to keep them organized.

### SUMMARY

It is an object of the present disclosure to provide a lead wire slide that eliminates or at least reduces the disadvantages of the related art. Specifically, it is the object of the present disclosure to provide a lead wire slide that is as handy as possible or practical even with many lead wires, cost-effective to produce, and/or enables to grab and move one particular lead wire as easily or reliably as possible.

This object is achieved by a lead wire slide for slidable accommodation of medical lead wires, comprising a base section that includes a plurality of through-holes that are each configured to accommodate one medical lead wire in a slidable manner and a connecting section that is provided on a side of the base section and configured to be connected to a complementary connecting section of another lead wire slide. The lead wire slide may be of unitary construction, i.e. made in one piece.

In the following, the lead wire slide may be referred to as a first lead wire slide. Another lead wire slide may be referred to as second, further, or (an)other lead wire slide.

Medical lead wire is to be understood as a lead wire or cable or line or wire that is included or provided to be used in any medical device or any medical application, and/or that transfers or is provided to transfer a current or data from any medical measurement. The medical lead wires may be electrode cables. The lead wires may include or be attached to an electrode, preferably for electroencephalography, or for example for electrocardiogram for defibrillators.

Slidable is to be understood as translatable at least in the direction in which the through-hole extends. Each through-hole may be configured to limit the movement of an accommodated (medical) lead wire in all directions except the direction(s) in which the through-hole extends (axially). In other words, if e.g. a through-hole has a cylindrical shape extending in an axial direction, it may be configured to accommodate a lead wire so that the lead wire cannot leave or may be limited in its movement in radial directions of the cylinder, but the lead wire can move along the axial direction. The term slidable also means that the wire can move with limited frictional engagement with the slider. The slide force to overcome friction may be below 10 N. Having some friction is advantageous in that the slide will not move to one end of the wire too easily, such as under the influence of gravity.

A through-hole is a passage all the way through the base section or lead wire slide from a first side to a second side.

The term side is used to describe a position or location or surface. A connecting section provided on a side may extend out of the plane to form a 3D-body or 3D-structure. Therefore, a section provided on a side may extend or protrude from that side.

The lead wire slide may be configured or designed in its function to be connected or be connectable to other lead wire slides and/or to be combined modularly, but can also be used individually.

The advantages of the technology disclosed herein are that the lead wire slide enables to untangle (medical) lead wires by sliding it along the lead wires. The lead wire slide enables to have the lead wires separated and organized, so that it is easy to grab one particular lead wire and to move it without moving the other lead wires and/or the lead wire slide. Thereby, for example, an electrode connected to one particular lead wire can be easily attached to a patient without moving the other lead wires or without e.g. unintentionally detaching already attached electrodes from a patient. For example, a doctor may hold in one hand the lead wire slide and a plurality of organized lead wires and may then grab one specific lead wire with the other hand and attach its electrode to a patient. Further, by using through-holes for the accommodation of the medical lead wires, it can be prevented that, when e.g. a lead wire is pulled, the lead wire is unintentionally detached from the lead wire slide. The lead wire slide may also for example be sold or shipped already in a state, in which lead wires are already accommodated in the through-holes and then the through-holes may prevent that the lead wires are detached from the lead wire slide during transportation.

By providing a lead wire slide with already accommodated lead wires, for example a doctor may be able to use the lead wires faster and easier, for example for an EEG measurement, and thereby time of the doctor and a patient may be saved. Further, the through-holes enable to use a single lead wire slide alone, since no other lead wire slide or part of one is needed to prevent the lead wires from unintentionally being detached or falling out of the through-holes or to form the through-holes. Furthermore, the connecting section enables to connect multiple lead wire slides to each other to practically form one (overall) lead wire slide with more through-holes for more lead wires. This modularity provides flexibility. For example, in case of EEG, the head of a patient is typically classified in three regions. Therefore, it may be advantageous to use three lead wire slides wherein each lead wire slide accommodates all the lead wires with electrodes for one region. This makes it easier for a doctor to see which lead wires (their electrodes) are provided for which or the same region. During attachment of the electrodes to a patient, the three lead wire slides then can be separated and used individually, simplifying the attachment. Before and/or after attachment, the lead wire slides can then be connected, to have one overall lead wire slide with one organized lead wire bundle. Further, this makes large amounts of lead wires better manageable, since then the one (overall) lead wire slide can be slided along the lead wires to organize them. The lead wire slide has overall a relative simple design, which makes it cost-efficient to produce, especially, if multiple or all connectable lead wire slides have an identical design. A further benefit of organizing the leads is found if the lead wires are applied to a patient undergoing Magnetic Resonance Imaging (MRI) in a Magnetic Resonance scanner. Such scanners use strong magnetic fields and are generally tunnel shaped. Lead wires arranged centrally in the scanner will take up less energy from the magnetic field compared to lead wires closer to the periphery, and with the lead wire slide it is possible to organize the lead wires and arrange the lead wires centrally in the scanner, thereby lowering the potential risk of harmful heating of the lead wires. Further, the lead wire slide may aid in straightening the lead wires and minimize the risk of loop formation of the lead wires. Such wire loops could potentially give rise to an electromagnetic coil effect negatively influencing the performance of the imaging.

The connecting section may be configured to form or to establish or to provide a form-fit and/or force-fit connection, e.g. a press-fit connection. The connecting section may be a plug. Alternatively, the connecting section may for example be a magnet, then a complementary connecting section (e.g. of another lead wire slide) may be made of metal and be magnetic, or vice versa. Alternatively the connecting section may be one part of a hook-and-loop-fastener, and then a complementary counterpart (e.g. connecting section of another lead wire slide) may be the respective other one.

The connecting section may be configured to establish a connection that is detachable. The connection may be establishable and detachable multiple times or repeatedly.

The connecting section may be configured to establish one (direct) connection to one other lead wire slide. One connecting section may be a section that is configured to connect to exactly one other lead wire slide at a time.

The other lead wire slide with the complementary connecting section may be of identical design, including their structure or construction. The lead wire slides may, however, be of unique and/or different designs.

The base section may have (essentially) a cuboid shape. The base section may have, respectively, two opposed (lateral) sides or surfaces, which are in the following referred to as a top and a bottom, a front and a back, and a left and a right (side or surface). These terms are used to simplify the description and depend on the point of view or the orientation.

The top and bottom may be parallel and flat surfaces, preferably rectangular. The front and back may be parallel and flat surfaces. The left and/or right side (surfaces) may be rounded (off) or curved or bent. At least one side may rounded or include a rounding and/or a recess, providing a gap between two connected (identical) lead wire slides to reach in to easily disconnect or separate them. The base section or lead wire slide may rounded off at the left and right sides, e.g. for better haptics. The lead wire slide may be (essentially) mirror-symmetric (in its design or construction) to a central plane between the front and back. There mirror plane is in the center between the front and back sides. The lead wire slide or base section has a front-back-axis (that is perpendicular to the front and/or back and runs through the center(s) or center point(s) of the front and/or back) as a first axis, a top-bottom-axes (that is perpendicular to the top and/or bottom and runs through the center(s) or center point(s) of the top and/or bottom) as a second axis, and a left-right-axis (that is perpendicular to the left and/or right and runs through the center(s) or center point(s) of the left and/or right) as a third axis, wherein these axes are orthogonal. Preferably, the lead wire slide is (rotationally) symmetrical in that, when the lead wire slide is rotated by 180° around the front-back-axis an identical lead wire slide is obtained. Additionally or alternatively, the same may account for a 180° rotation around the top-bottom-axis and/or the left-right-axis.

Each through-hole may run through the lead wire slide or base section in a straight line. The through-holes may also be staggered, potentially aligned along parallel straight lines.

At least one (preferably all) through-hole(s) include(s) a fillet or chamfer (preferably one at both ends of a through hole) for guiding a medical lead wire inside or through and/or for smoothening an edge for protecting a medical lead wire (that may be accommodated in the through-hole).

Preferably, the through-holes run in parallel to each other.

All through-holes may run from a first surface of the base section, preferably a flat lateral surface, to a second surface of the base section, preferably a flat lateral surface. The first flat lateral surface and the second flat lateral surface may be parallel.

The first flat lateral surface may be the front or front side or front surface and the second flat lateral surface may be the back or back side or back surface (of the base section or lead wire slide). Preferably, the through-holes run perpendicular to the front and back.

The through-holes may be arranged in the base section next to each other on a line or row. Alternatively, the through-holes, especially in case of a large number of through-holes, may be arranged in rows and columns, orthogonal or staggered.

The lead wire slide may include two to ten, preferably seven to ten, particularly preferably nine through-holes.

The through-holes may all have a cross-section of the same shape and size. However, the through-holes may have cross-sections of different sizes or shapes, for example for differently sized or shaped lead wires.

Preferably, the through-holes have each circular cross-sections, preferably corresponding to the medical lead wires that usually also have circular cross-sections. The through-holes may for example each have a circular cross-section with a diameter larger than 1.7 millimetres. It is to be understood that, for example in case of a circular cross-section, a diameter of one particular through-hole may be larger than a diameter of a particular or corresponding or associated medical lead wire (that may be accommodated in this through-hole in a slideable manner), so that they may form a clearance fit, so that the medical lead wire may slide (easily) in the through-hole.

Preferably, the base section, and particularly preferably the whole lead wire slide is one piece, i.e. is an integral part.

Preferably, the lead wire slide contains or includes no magnetic materials, This makes it then possible to use it in an magnetic resonance imaging (MRI) scanner.

Preferably, the lead wire slide is made of polymer material, particularly preferably Polypropylene (PP). Without ruling out other materials PP is found to have some favourable features, such as low cost, and semi-rigid mechanical properties to enable click-able connections without being too rigid, so it is friendly to the patient. Polypropylene is also easily recyclable, thereby lowering the environmental impact and improving sustainability. Use of bioplastics, such as bio-PP, could further improve sustainability.

Preferably, the lead wire slide is produced by injection moulding. Such a lead wire slide may then be produced cost-efficiently.

Preferably, the connecting section is configured to form a force-fit connection with or to a complementary connecting section of another lead wire slide. Preferably, it is configured to establish or form and release the force-fit connection when a (resulting) force between 4.5 to 10 N is applied. In other words, preferably such a force is necessary to connect and disconnect two lead wire slides. For example, the connecting sections of two lead wire slides may be configured to disconnect the lead wire slides, when they are pulled apart with a resulting force of 4.5 to 10 N, for example when on each lead wire slide a (separating) force of 2.25 to 5 N is applied. Therefore, it may be prevented that two connected lead wire slides fall apart by themselves, while on the other hand assuring that the connected lead wire slides can be disconnected without too much force needed, and preferably without the need for tools. As used herein, a force between A and B or a force of A to B includes A and B.

Preferably, the lead wire slide includes a further or second connecting section. Preferably, this further or second connecting section is provided on a side of the base section opposite to the side of the (first) connecting section. Preferably, the (first) connecting section is provided on the top of the base section and the further or second connecting section is then provided at the bottom. Then a first other lead wire slide may be connected to the top by the (first) connecting section and a second other lead wire slide to the bottom by the further or second connecting section. Preferably, the connecting section is provided centrically or towards the center of the top surface. Preferably, the further connecting section is provided at the same position in the bottom surface, i.e. opposed to the connecting section. Thus, lead wire slides may be stackable (without an offset). The disclosure is not limited to this, e.g. the connecting section may be at the top and the further connecting section at the left side or surface. The lead wire slide may include even more connecting sections, for example, the lead wire slide may include the first, the second, a third, and a fourth connecting section: One at the top, one at the bottom, and one at the left and one at the right. Then four other lead wire slides may be connectable to the lead wire slide, one at the top, one at the bottom, one at the left and one at the right.

Preferably, the (first) connecting section and the further (second) connecting section are complementary counterparts (of each other). Then a preferably identical first other lead wire slide may be connectable to the (first) connecting section and a preferably identical second other lead wire slide may be connectable to the further connecting section. In other words, then identical lead wire slides may be stackable and connectable to each other, similar to Lego® bricks.

Preferably, opposed connecting sections are complementary counterparts (of each other). The lead wire slide may include the first, the second, a third, and a fourth connecting section. Preferably, two connecting sections of the lead wire slide are complementary counterparts, respectively. In other words, preferably two connecting sections can be interpreted as a pair, wherein the connecting sections of one pair are complementary counterparts of each other.

The connecting section and the further connecting section may not be complementary counterparts. For example, when every connecting section of the lead wire slide is individual, only one particular counterpart of one particular other lead wire slide may be connectable. Thereby, it may be possible to determine, which particular other or further lead wire slide is connectable. This may make it possible to determine orders, i.e. in which order a plurality of lead wire slides is connectable. For example, a first lead wire slide may only be connectable to a second lead wire slide, but not to a third, wherein the third is also connectable to the second one.

Preferably, the connecting section and its counterpart(s) (preferably, the further connecting section is a counterpart, and preferably, at least one other or the (an)other lead wire slide includes a connecting section that is a counterpart) are designed or configured so that they may or could be connected to each other. As described above, this may be e.g., a magnetic connection or in form of a hook-and-loop-fastener. Preferably, the connecting section and its counterpart(s) are designed or configured to form a force-fit and/or form-fit connection, preferably an oversize-fit or press-fit. A form-fit may have advantage that it is easily formed or established or connected and/or disconnected (no force needed), wherein a force-fit may have the advantage that it may be prevented that a connection is released or disconnected unintentionally or by its own. A form-fit connection may preferably be provided in a (connection) direction perpendicular to the direction into which the through-holes run or to the direction into which the lead wire slide may be slided (along the lead wires), so that when an assembly of connected lead wire slides is moved along the (medical) lead wires, the connection of the lead wire slides is not unintentionally released.

For example, the connecting section may include a protrusion and its counterpart(s) may include a complementary recess, wherein the protrusion fits into the recess. For simplicity, in the following (connecting) sections or portions that protrude (from the lead wire slide, the base section or the connecting section) are referred to as male (connecting) portions, and correspondingly (connecting) sections or portions that recede or sink or are recessed or are able or provided or configured to accommodate a male (connecting) portion are referred to as female (connecting) portions. Female (connecting) portions may be a subtraction of or negative extrusion of or extrusion of a 3D-body or structure from or inside another 3D-body or structure, e.g. a subtraction of a male (connecting) portion from the base section. A female connecting portion may result out of one or multiple protrusion that provide(s) or form(s) a space (in between) into which something (e.g. a male connecting portion) may be put, arranged, or plugged into.

Preferably, the connecting section includes (at least) a male connecting portion and its counterpart(s), e.g. the further connecting section and/or connecting section of another lead wire slide, includes (at least) a female connecting portion or vice versa, wherein the male and female connecting portions are configured to form a force-fit and/or form-fit connection with the respective other one (of another lead wire slide, preferably of identical design).

Preferably, the female connecting portion includes an inlet bevel or chamfer that is configured to guide a/the male connecting portion into the female connecting portion. Preferably, the male connecting portion has a (corresponding or complementary) inlet bevel or chamfer that is configured to guide the male connecting portion into a/the female connecting portion.

The male connecting portion may (essentially) have a cuboid protruding shape (preferably with inlet bevels or chamfers). The female connecting portion may (essentially) have a cuboid receding shape (preferably with inlet bevels or chamfers).

Alternatively, for example, the male connecting portion may be a cylinder or cylindrical protrusion and the female connecting portion may be a cylinder or cylindrical recess. Alternatively, for example, the male connecting portion may have the shape of a dovetail and the female connecting portion may then be a complementary recess or groove, so that the male and female connecting portion are able to or configured to provide or form a dovetail joint. The dovetail joint is usually a form-fit, but may be a press-fit, if the male connecting portion is for example oversized and pressed into the female connecting portion. As described above, a/the male and a/the female connecting portion(s) is/are preferably complementary counterparts.

Preferably, the male connecting portion and/or the female connecting portion is/are bendable or deformable under force, so that they may be pressed into each other by force and form then an interference fit or press-fit or friction fit. In other words, preferably the male connecting portion and the female connecting portion are configured to form a press-fit or friction fit (with the respective other one, preferably of another lead wire slide, preferably of identical design).

Preferably, the connecting section includes (at least) one male connecting portion and one female connecting portion and its counterpart(s), e.g. the further connecting section, includes also (at least) one female connecting portion and one male connecting portion, wherein preferably the male connecting portions are counterparts to the female connecting portions (and vice versa). This enables to form or establish a double connection between the connecting section and its counterpart(s), and thus for example, it may be prevented that the connecting section of the lead wire slide (and the whole lead wire slide) is rotatable relative to (its counterpart of) another lead wire slide, when they are connected with each other.

Preferably, one of the female connecting portion and the male connecting portion includes a preferably pin-shaped protrusion and the other one includes a complementary preferably slot-shaped recess, wherein the preferably pin-shaped protrusion and the preferably slot-shaped recess are configured to snap together with the respective other one (of another lead wire slide, preferably of identical design), when the female connecting portion and the male connecting portion are or get or become connected. Preferably, the preferably pin-shaped protrusion and the preferably slot-shaped recess are configured to form a form-fit. Thus, preferably additionally to a friction fit as described above, two connecting sections (a connecting section and its counterpart(s)) may also be configured to form or establish a form-fit. The preferably pin-shaped protrusion, preferably slot-shaped recess, male connecting portion and/or female connecting portion may be bendable or pressable, so that the preferably pin-shaped protrusion and preferably slot-shaped recess are configured to snap into each other. For example, their material, preferably a polymer, may be (a little bit) bendable, or for example a spring may be used. Preferably, the preferably pin-shaped protrusion and preferably slot-shaped recess are configured to be connectable and detachable (under force) to and from each other, preferably for multiple times.

The female connecting portion or the male connecting portion may include multiple preferably pin-shaped protrusions or preferably slot-shaped recesses. Preferably, one connecting section includes two preferably pin-shaped protrusions and two complementary preferably slot-shaped recesses for double securing the connection. Preferably, the connecting section includes one male and one female connecting portion, wherein one of the male connecting portion and the female connecting portion includes two preferably pin-shaped protrusions and the other one two corresponding or complementary preferably slot-shaped recesses. Preferably, then the/a counterpart of the connecting section (e.g. connecting section of another lead wire slide and/or a connecting section of the lead wire slide on an opposite surface) includes also one male and one female connecting portion (as counterparts to the ones of the connecting section) that include two preferably pin-shaped protrusions and/or preferably slot-shaped recesses, complementary to their respective male or female counterpart connecting portion.

The purpose of a through-hole may be to ensure that a medical lead wire accommodated in the through-hole cannot leave the through-hole (in another direction then the one(s) into which the through-hole extends, i.e. for example in radial directions) (at least not on its own, e.g. cannot fall out). A through-hole may include open portions, i.e. not be fully surrounded by material or not be fully closed (along its perimeter), and/or may be (similar to) a groove.

Preferably, the lead wire slide includes smoothed edges and/or corners. Particularly preferably, all edges and/or corners of the base section or the lead wire slide are smoothed, that means they are not or less sharp. In other words, the lead wire slide preferably includes no or only a small number of sharpened edges and/or corners. This means that the lead wire slide may provide better haptics, a risk of injury may be decreased, and/or it may prevent that a sharp edge may damage a lead wire. Smoothed may be understood as that the corner and/or edge may be provided with a fillet, chamfer, curvature, rounding and/or be rounded off. Alternatively, only some or at least one corner or edge may be smoothed, for example one/the ones that is/are provided or placed the furthest out or at the outer periphery or a/some/the outer edge(s) (pointing outwards).

Preferably, the base section of the lead wire slide may include at least one groove, that is configured to accommodate one medical lead wire in a slidable manner, wherein the medical lead wire can be pushed in or out of the groove. Preferably, the groove includes a bendable portion, that enables inserting a lead wire under force, but prevents the lead wire from leaving the groove on its own, i.e. when no force (of a user) is applied. Preferably, the lead wire slide includes two grooves, preferably at its outer periphery and/or one at/towards the left and/or the right side (surface) further out (side) than the through-holes. Due to such a groove, an additional lead wire can be added to the lead wire slide and can, if not needed, easily be removed (taken out of the groove).

Further, the present disclosure relates to an assembly group including a first lead wire slide or the lead wire slide and a second lead wire slide or another or a further lead wire slide, preferably one or each as described above, wherein the first lead wire slide and the second lead wire slide are connected or connectable by connecting, preferably as described above, a/the connecting section of the first lead wire slide and a/the connecting section of the second lead wire slide that are complementary counterparts and configured to be connected to each other, preferably as described above.

In other words, the lead wire slides are preferably modular and can be stacked or connected or attached to each other. The first and/or second lead wire slide may have features as they are described above (e.g. for the lead wire slide).

Preferably, the first lead wire slide and the second lead wire slide are identical or of identical design or construction. They may have a different design, e.g. the first lead wire slide may have only a male connecting portion and the second lead wire slide may only have a complementary female connecting portion. The assembly group may include more than two lead wire slides, for example three. In particular, if lead wires (for which that assembly group may be provided for) may be classified or classifiable in different categories or groups, the assembly group preferably includes one lead wire slide for each category, so that all lead wires of one category may be accommodated in the same one lead wire slide. If e.g. the lead wires are attached to electrodes for EEG applications, and the electrodes are classified for example in three groups for three regions on a patient's head, then preferably the assembly group includes three lead wire slides, wherein each is provided to accommodate the lead wires with the electrodes of one of the three groups.

Preferably, the connecting section of the first lead wire slide and the connecting section of the second lead wire slide are connectable as counterparts as described above.

Preferably, the first lead wire slide and the second lead wire slide are connected or connectable so that all through-holes of the first lead wire slide and the second lead wire slide run in parallel to each other.

Preferably, the first lead wire slide and the second lead wire slide are connected or connectable so that the assembly group is handy and can be used or hold as one unit.

Preferably, the first lead wire slide and the second lead wire slide are connected or connectable so that they cannot be rotated and/or moved translationally relative to each other (except for when a force to disconnect them is applied).

Preferably, the assembly group includes a third lead wire slide connected or connectable to the first lead wire slide and/or the second lead wire slide. There may also be provided more than three lead wire slides connected or connectable to each other, e.g. four, five, six, seven, etc.

Further, the present disclosure relates to a medical system comprising a lead wire slide, preferably as described above, or an assembly group, preferably as described above, and further comprising a medical lead wire, preferably as described above, wherein the medical lead wire is accommodated in a through-hole of the lead wire slide or in a through-hole of the first or the second lead wire slide of the assembly group in a slidable manner, preferably as described above.

Preferably, one end of the medical lead wire is connected to an electrode (preferably for EEG measurements) and the other end is connected to a connector (configured or suitable for a connection to a medical device, preferably an EEG device). Connected may include (here) attached and/or mounted. The connector may e.g. be a hub bundling multiple medical lead wires.

Preferably, the lead wire slide or the assembly group is provided along the medical lead wire in between the electrode and the connector and/or the ends of the medical lead wire.

The electrode and the connector may include larger (cross-) sections than the one of the through-hole, in which the medical lead wire is accommodated. Preferably, the lead wire slide or the assembly group is (e.g. thereby) prevented from (unintentionally) sliding off or being detached or removed from the medical lead wire (without detaching the electrode or connector first).

Preferably, a plurality of lead wires are provided which are accommodated in the through-holes of the lead wire slide or the plurality of lead wire slides.

### BRIEF DESCRIPTION OF THE DRAWINGS

The technology disclosed herein will be described in more detail below with reference to the following figures. The figures illustrate embodiments, variations and examples of the technology disclosed herein to facilitate the understanding of a person of ordinary skill in the art and are not to be construed as limiting the scope of the claims.

FIG. 1 is a perspective view of an embodiment of a lead wire slide according to the present disclosure.

FIG. 2 is another perspective view of the lead wire slide of FIG. 1.

FIG. 3 is a perspective view of an assembly group including stacked lead wire slides.

FIGS. 4, 5, and 6 are side views of the lead wire slide of FIG. 1 with an additional feature.

FIG. 7 is a cross-sectional view of the lead wire slide of FIGS. 1 and 3.

FIG. 8 is a cross-sectional view of the lead wire slide of FIGS. 1 and 3 with an additional feature.

FIG. 9 is a sectional view of a side of the lead wire slide of FIG. 8.

FIG. 10 is a perspective view of a lead wire slide including the features shown in FIG. 1 and additional features.

FIG. 11 is a side view of the lead wire slide of FIG. 10.

FIG. 12 is a bottom view of the lead wire slide of FIG. 10.

FIG. 13 is a front view of the lead wire slide of FIG. 10.

FIG. 14 is another side view of the lead wire slide of FIG. 10.

FIG. 15 is a side view of another lead wire slide according to the present disclosure.

FIG. 16 is a side view of an assembly group including stacked lead wire slides similar to the lead wire slide of FIG. 15.

FIG. 17 is a side view of a further lead wire slide according to the present disclosure including features of the lead wire slides of FIGS. 1 and 15.

FIG. 18 is a perspective view of a medical system 38 including a lead wire slide.

### DETAILED DESCRIPTION OF SELECTED EMBODIMENTS

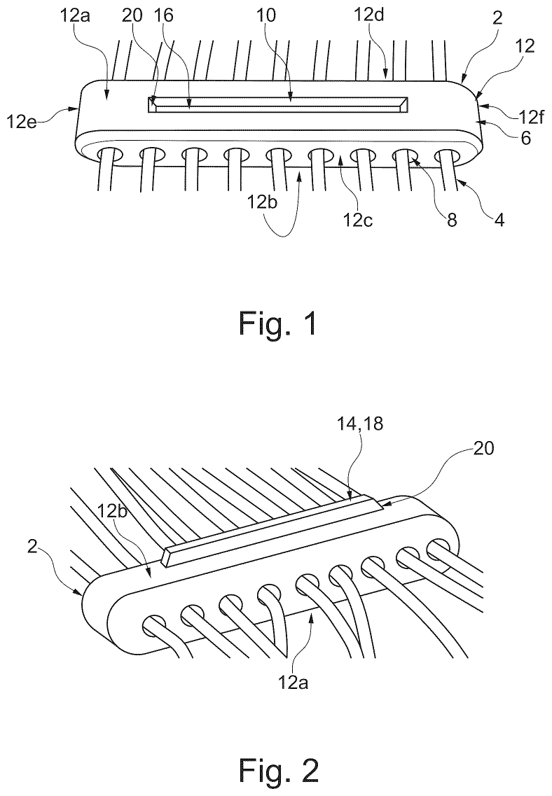

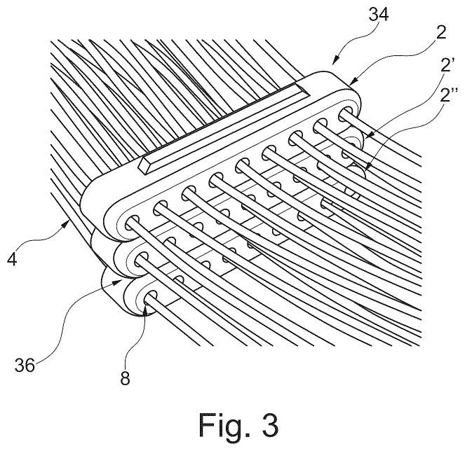

FIG. 1 shows a lead wire slide 2 for slidable accommodation of medical lead wires 4. For illustration purposes, FIG. 1 shows medical lead wires 4. Generally, the lead wire slide 2 is configured to mate, and form a stack, with another lead wire slide 2. The lead wire slides may comprise a set of mating features, two sets of mating features or even more sets of mating features, each set of mating features comprising a feature on one lead wire slide and a mating feature on the other lead wire slide. The lead wire slide 2 includes a base section 6 that includes a plurality of through-holes 8 that are each configured to accommodate one medical lead 4 wire in a slidable manner. Here, the lead wire slide includes nine through-holes 8, that are positioned adjacent/next to each other on a straight line with their longitudinal extents in parallel. The lead wire slide 2 further includes a first connecting section 10 that is provided on a side or surface 12 of the base section and configured to be connected to a complementary connecting section 10′ of another lead wire slide 2′ (see FIG. 3). Numeral 12 refers, generically, to a side or surface of the lead wire slide 2, while specific sides are denoted by the numeral 12 and a respective letter.

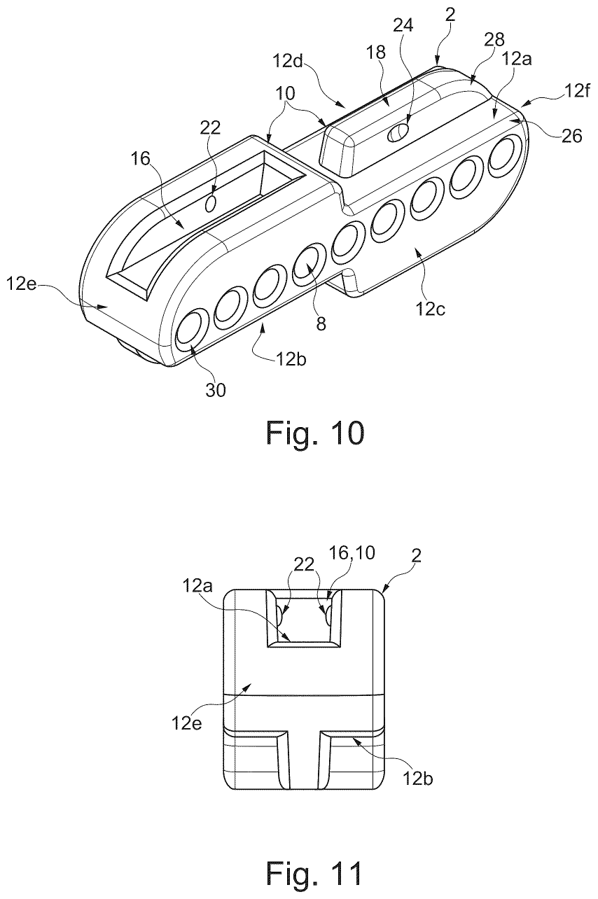

As described with reference to FIGS. 1 to 9, the lead wire slide may comprise a first connecting section comprising a single connecting portion. The single connecting portion may be male or female. The lead wire slide may also comprise a second connecting section comprising a single connecting portion. The single connecting portion of the second connecting section may be male or female and be complementary of the single connecting portion of the first connecting section. Furthermore, as described with reference to FIGS. 10 to 14, the first connecting section may comprise two connecting portions, one female and the other male. Analogously, the second connecting section may comprise two connecting portions, one female and the other male, and both complementary to the connecting portions of the first connecting section.

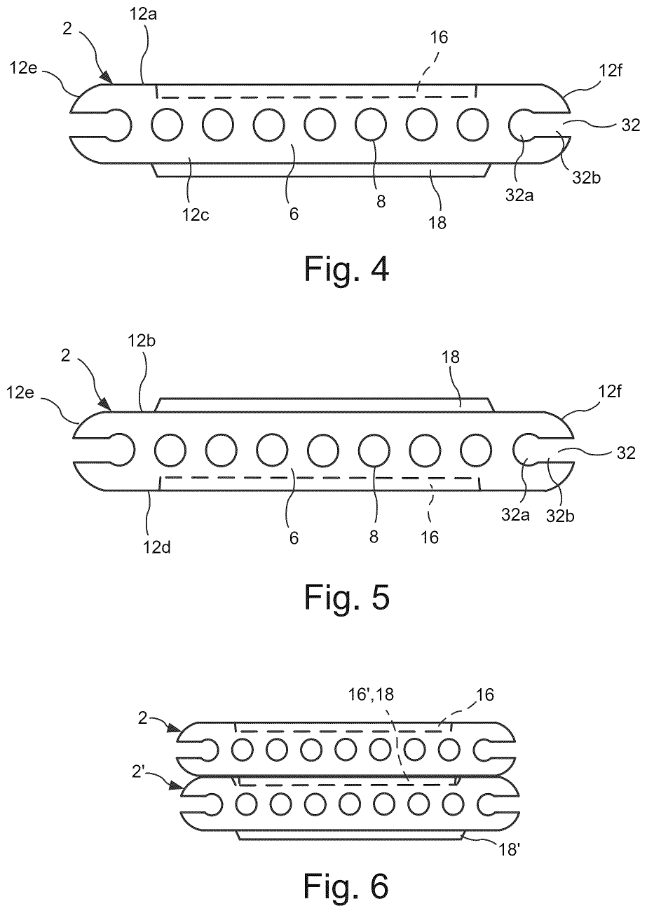

The first connecting section 10 is provided on a side 12 that is referred to as the first, or top, side 12a for a better description and visualization. The base section 6 has an essentially cuboid structure, wherein the through-holes 8 run straight and in parallel to each other from a front 12c to a back 12d (side) that are flat lateral surfaces opposed to each other. The lead wire slide 2 may be the same when viewed from the front 12c and back 12d. In other words, it may be symmetrical (with a mirror plane as a middle plane between the front and back). The left and right sides 12e, 12f (the ends/sides along a longitudinal extent of the lead wire slide 2) are rounded off or in other words these sides or surfaces or ends are bent or curved. This provides better haptics. Having smooth curves and avoiding sharp edges improves ergonomics for both the patient and the health care professional. One or both of the outermost through-holes 8 may be formed as an open groove, or open slot, 32 opening towards the left or right sides 12e, 12f, thereby allowing one or two medical lead wires 4 to be fitted in or removed from lead wire slide 2, as shown in FIGS. 4-6 and as will be discussed below with reference to FIG. 15.

The first connecting section 10 may be provided centrically and symmetrically in the first side 12a. The first connecting section 10 seen in FIG. 1 comprises a recess or female connecting portion 16. The female connecting portion 16 is configured to accommodate/mate with a complementary counterpart, a protrusion or male connecting portion 18′ of a lead wire slide 2′ (see FIG. 3). The female connecting portion 16 and the male connecting portion 18′ comprise a set of mating features. Each lead wire slide may comprise a set of mating features, which allows the lead wire slide to mate with either component of the set of another lead wire slide. Providing identical lead wire slides reduces inventories and simplifies the medical procedure because a practitioner does not have to select lead wire slides based on their features. The female connecting portion 16 has (essentially) a cuboid shape including in this case two inlet bevels or chamfers 20. Alternatively, the first connecting section 10 may be offset right to left, left to right, front to back or back to front so that it is not centrically and symmetrically in top 12a. Providing an offset removes a degree of freedom when stacking the lead wire slides and may be used to provide an orientation direction. Additionally or alternatively, the first connecting section 10 may be obliquely positioned relative to the middle plane, whether positioned centrally or offset.

FIG. 2 shows a bottom, or second, side 12b view of the lead wire slide 2. The bottom side 12b includes a second connecting section 14 that is in this case complementary (counterpart) to the first connecting section 10 provided on the top side 12a. This connecting section 14 comprises a protrusion or male connecting portion 18. The male connecting portion 18 is provided on the bottom 12b at a position corresponding or similar to the position of the female connecting portion 16 on the top 12a, in other words opposed to it. Thereby the ends of the stacked lead wire slides may be aligned. The male connecting portion 18 also has (essentially) a cuboid shape including in this case two inlet bevels or chamfers 20. The male connecting portion 18 and the female connecting portion 16 are configured to be connected to one of the other of another, exemplary identical, lead wire slide 2′ (see FIG. 3) and to form/or establish a force-fit or press-fit connection. In other words, the connecting sections 10 and 14 (16 and 18) are configured to provide a connection to other lead wire slides. For example, another (identical) lead wire slide 2′ is connectable with its bottom 12b to the top 12a of this lead wire slide 2 by connecting the second connecting section 14′ having a male connecting portion 18′ of the other lead wire slide 2′ to the connecting section 10 having a female connecting portion 16 of this lead wire slide 2. Also, for example another (identical) lead wire slide 2′ is connectable with its top 12a to the bottom 12b of this lead wire slide 2 by connecting the first connecting section 10′ having a female connecting portion 16′ of the second lead wire slide 2′ to the second connecting section 14 having a male connecting portion 18 of this lead wire slide 2. In other words, the lead wire slide 2 is configured to be connected or stacked with or to others.

The design of the lead wire slide 2 enables a cost-effective production and provides good haptics. The lead wire slide 2 may be made of a polymer material, in particular Polypropylene (PP), preferably with a high proportion of bio-PP, and includes no metal. Therefore, it may be produced cost-efficiently, is usable in MRI-scanners, and may reduce the carbon footprint by facilitating recycling. A lead wire slide 2 may be injection molded as single-piece part.

FIG. 3 shows an assembly group 34 of in this case three lead wire slides 2, 2′ and 2″. The assembly group 34 comprises a first lead wire slide 2′ and a second lead wire slide 2′ that are identical to the lead wire slide 2 of FIG. 1. The first lead wire slide 2 and the second lead wire slide 2′ are connected or connectable by connecting the connecting section 10 of the first lead wire slide 2 and the connecting section 10′ of the second lead wire slide 2′ that are complementary counterparts and configured to be connected to each other. Here, exemplarily, the connecting section 10 of the first lead wire slide 2 is the female connecting portion 16 in the bottom 12b of the first lead wire slide 2 and the connecting section 10′ of the second lead wire slide 2′ is the male connecting portion 18 on the top 12a of the second lead wire slide 2′. For illustration purposes, medical lead wires 4 are also shown. The three lead wire slides 2, 2′ and 2″ are connected or connectable so that all through-holes 8 of the first lead wire slide, the second lead wire slide and the third lead wire slide run in parallel to each other.

At least one side 12 of each lead wire slide 2, 2′, 2″ is provided with a recess or rounding or curvature or is rounded (off) or curved or bent, providing a gap 36 between two connected lead wire slides. Here, the left 12e and right sides 12f are rounded, so that there is a gap 36 (created) into which one can grab in order to easily separate two connected lead wire slides.

To provide a slidable accommodation of a medical lead wire 4 in the lead wire slide 2 the through-holes 8 are generally slightly larger than the medical lead wire 4. As an example, the through-holes 8 may have a diameter, which is 0.4 to 0.8 mm larger than the diameter of the medical lead wire 4, such as 0.5-0.7 mm larger.

FIGS. 4, 5, and 6 illustrate an additional feature of the lead wire slide 2, referred to as an “end slot.” In these examples the furthest through-holes 32a on the right and left sides are open-sided and are connected to a narrow slot 32b that has a gap smaller than a diameter of the open-sided hole, forming a through-slot, or end slot, 32. The medical lead wires 4 may be threaded through the closed-sided through-holes 8 except when the ends of the medical lead wires 4 are attached to something larger than the front or back area of the closed-sided through-holes 8, because in that case they lack a free end that could be threaded through a closed-sided through-holes 8. The through-slot 32 enables positioning a medical lead wire 4 laterally, through the open end of the through-slot 32, instead of by threading it. The narrow slot 32b has a gap that may be the same or slightly smaller than the diameter of the medical lead wire so that after positioning the medical lead wire will remain in the open-sided through-hole 32a and will not unintentionally slip out of the end slot. The end slots may be identical, as shown. Alternatively, the sizes of the end slots may differ to accommodate different wire sizes. In FIG. 6 the stacked lead wire slides 2, 2′ comprise open-sided through-holes 32 in addition to closed-sided through-holes 8. The first lead wire slide 2 is stacked with the second lead wire slide 2′, wherein the male and female connecting sections are mated. In all other respects the lead wire slides 2,2′ shown in FIGS. 4-6 are the same as shown in FIGS. 1 and 2. The lead wire slides 2 shown in FIGS. 1-6 may comprise additional features as described below. The end slot may be substituted by an open-sided hole, as shown in FIG. 15, by omitting the narrow slot 32b. The open gap on the side of the open-sided hole may be equal or slightly smaller than a diameter of the wire. It should be clear from the functionality of the through-holes 8 that the term “wire” is inclusive of the jacket or insulation of the electrical conductor within it, and that the conductor may comprise one or more wire strands.

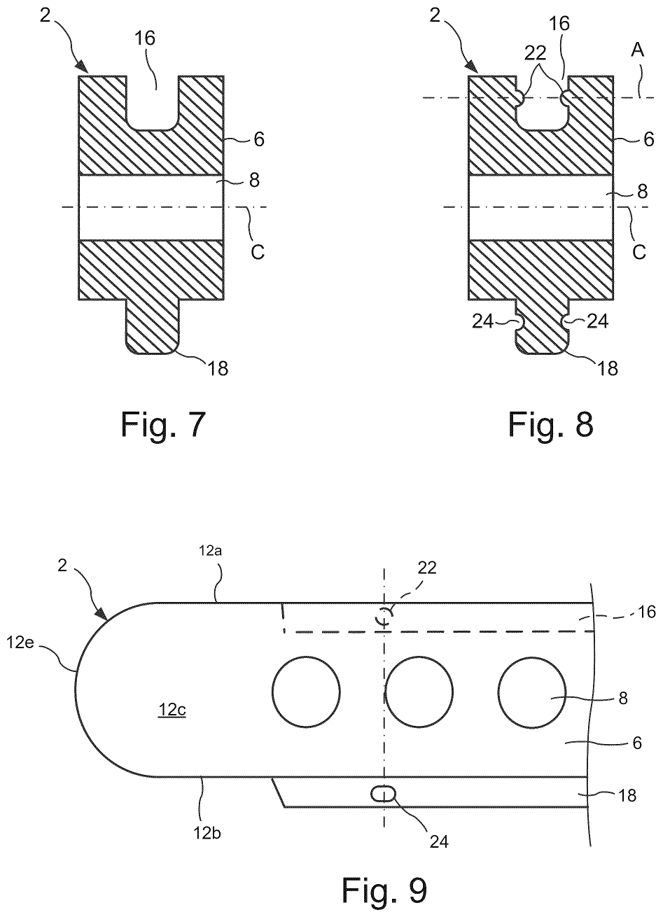

FIG. 7 is a schematic cross-section of the lead wire slide 2 through a closed-sided through-hole 8. A centerline C passes through the center of the through-hole 8 and defines the longitudinal extent of the through-hole 8. A set of mating features is shown, comprising the female connecting portion 16 and the male connecting portion 18.

FIG. 8 is a schematic cross-section of the lead wire slide 2, through a closed-sided through-hole 8, illustrating another additional feature of the lead wire slide 2, comprising one or more protrusions 22 extending inwardly from opposing sides the female connecting portion 16 and one or more recesses extending outwardly from opposing sides the male connecting portion 18. FIG. 9 is a schematic sectional view of the lead wire slide 2 of FIG. 8. A set of a protrusion and a recess form a snap-connection. More than one snap connection may be provided in a set of connecting sections. Optionally, the protrusions will comprise pairs of concentric protrusions, as shown. Alternatively, the protrusions may be axially offset and thus not concentric protrusions (not shown). The protrusions 22 and recesses 24 are further described with reference to FIG. 10. A centerline A passes through the centers of the protrusions 22 to illustrate their concentricity. Alternatively, only one protrusion and only one recess are provided, which may be referred to as a second set of mating features or as a set of mating features of a different type from the first set of mating features comprised by the connecting portions 16,18, on the top and on the bottom sides of the lead wire slide 2. Multiple sets of the second mating features (e.g. snap connections) may be provided to further tailor the forces required to stack the lead wire slides 2. Either or both of the protrusion and recess may comprise a portion of a sphere (circular cross-section), an ovoid (oval cross-section), or other shapes configured to easily slide into one another. The resiliency of the polymer used to mold (i.e. injection mold) the lead wire slide 2 enables the opposing walls to flex outwardly and then return to their original shape when the lead wire slides 2 are stacked.

FIGS. 10-14 illustrate additional features provided on the lead wire slide 2 of FIG. 1. Whereas the lead wire slide 2 of FIG. 1 comprises a first set of mating features, the additional features illustrated in FIGS. 10-14 include the first set of mating features, a second set of mating features, a third set of mating features, and a fourth set of mating features. Optionally, the third and fourth set of mating features, comprising snap connections, may be omitted. The second set of mating features comprises another set of male/female connecting portions, so that the top side of the lead wire slide 2 comprises a connection section including a female connecting portion and a male connecting portion, and so that the bottom side of the lead wire slide 2 also comprises a connection section including a female connecting portion and a male connecting portion. As shown, the male and female connecting portions include the protrusions and recesses discussed above, which form the snap connections. In all the examples herein, the positions of the protrusions and recesses may be switched such that the female connecting portion includes a recess and the male connecting portion includes a protrusion.

FIG. 10 shows on the top 12a a connecting section 10 configured to be connected to a complementary connecting section 10′ of another lead wire slide 2′. The connecting section 10 includes a female connecting portion 16 and a male connecting portion 18. Thereby, the connecting section 10 is configured to establish a double connection with a complementary counterpart. The female connecting portion 16 (of the connecting section 10) includes in this case two preferably pin-shaped protrusions 22. The male connecting portion 18 includes two complementary, preferably slot-shaped recesses 24. The female connecting portion 16 and male connecting portion 18 are complementary counterparts that would fit into each other to form a force-fit connection, wherein the preferably pin-shaped protrusions 22 and preferably slot-shaped recesses 24 are also counterparts and would snap into each other additionally forming a form-fit connection. Provision of engaging protrusions and recesses (preferably pin-shaped protrusions and preferably slot-shaped recesses) makes it easier to provide a connection with a tailored force. Edges and/or corners 26 of the lead wire slide 2 are smoothed by fillets 28 and/or chamfers 30 to provide favourable ergonomics and a pleasing design. The chamfers 30 may have a curvature of for example 0.3 mm, whereas the fillets 28 may have a curvature of for example 0.4 mm, while some outer edges may have a somewhat larger curvature, such as 0.8 mm. In a variant, one or both of the outermost through-holes 8 may be provided as grooves 32 opening towards the respective ends 12e, 12f to enable a medical lead wire 4 to be inserted or removed from the through-hole 8, as will be discussed below with reference to FIG. 15.

FIG. 11 shows the lead wire slide 2 of FIG. 10 from the left, showing the left side 12e. The female connecting portion 16 of the connecting section 10 provided on the top 12a is shown with its preferably pin-shaped protrusion 22.

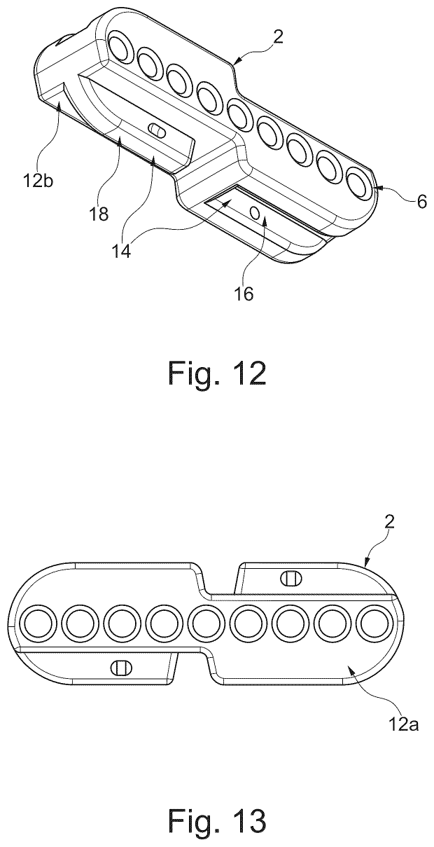

FIG. 12 shows the lead wire slide 2 of FIG. 10 from the bottom, showing the bottom (side) 12b. On the bottom 12b of the base section 6 is a further or second connecting section 14 provided, that (also) includes a female connecting portion 16 and a male connecting portion 18. The second connecting section 14 is the mirrored first connecting section 10. The first connecting section 10 is a complementary counterpart of the second connecting section 14, so that the top 12a of this lead wire slide 2 is connectable to the bottom 12b of an identical other lead wire slide 2′ and vice versa.

FIG. 13 shows the lead wire slide of FIG. 10 from the front, showing the front (side) 12a. This figure shows the symmetries of the lead wire slide 2. For example, when rotating this lead wire slide 2 by 180° around an axis perpendicular to the front 12a or back 12b surface, an identical lead wire slide would be obtained.

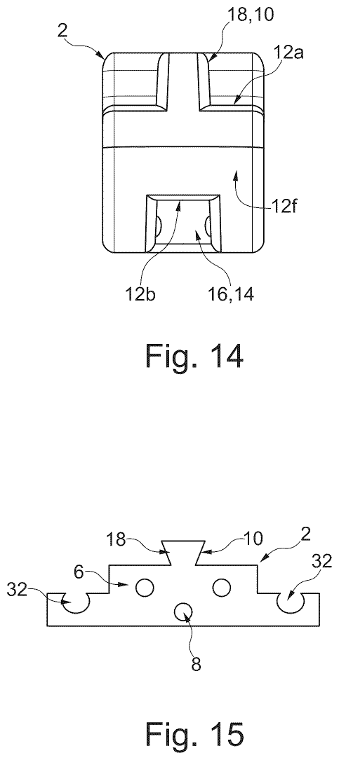

FIG. 14 shows another side view of the lead wire slide of FIG. 10 showing the right side 12f. FIG. 11 and FIG. 14 visualize that the top side 12a is configured to be connected to a/the bottom side 12b and vice versa, or in other words, that they are complementary counterparts.

FIG. 15 shows a different design of a lead wire slide 2. This lead wire slide 2 includes through-holes 8 and additionally grooves 32. The grooves 32 are each configured to accommodate one medical lead wire 4 in a slidable manner. A medical lead wire 4 can (under force) be inserted or pushed into or taken out or pulled out of a groove 32. This lead wire slide 2 includes (only) one connecting section 10, here a male connecting portion 18 in shape of a dovetail, that in this case is configured to form a form-fit connection with a complementary connecting section 10′ of another lead wire slide 2′ (not shown in this figure).

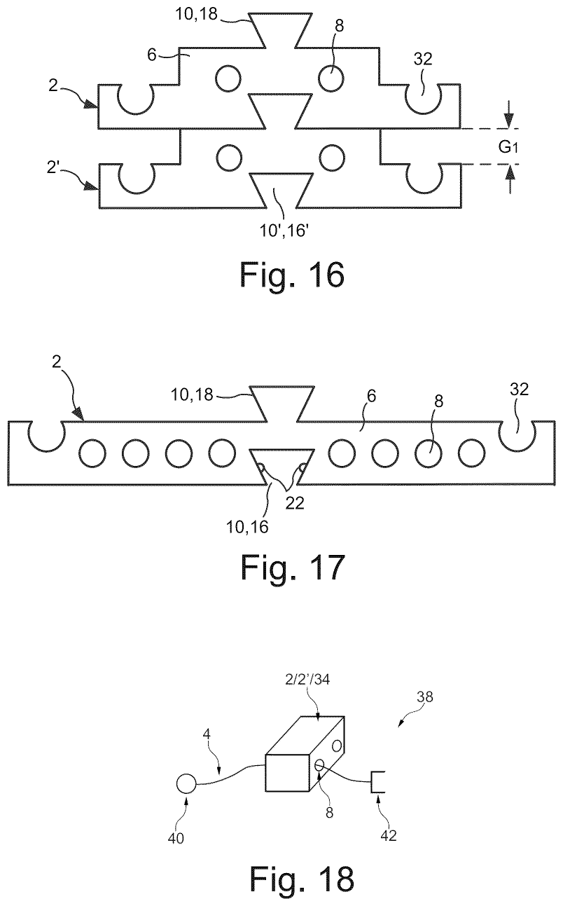

FIG. 16 illustrates how the dovetail joint is made with two, and potentially more, lead wire slides 2, with a design similar of that of FIG. 15, where the grooves 32 are open toward the top side of the lead wire slides 2. A gap G1 between the lead wire slides 2 allows placement f wires in the grooves 32 after the lead wire slides 2 have been stacked. FIG. 17 shows a similar design, except it includes, optionally, a second connecting section 10, the protrusions 22, and, although not visible, the recesses 24. The gap G1 is removed so that wires have to placed in the grooves 32 before the lead wire slides 2 are stacked.

FIG. 18 shows a medical system 38 comprising a lead wire slide 2, e.g. as in any one of FIG. 1, 2, 4, 5, or 7-17, or an assembly group 34, for example as shown in FIGS. 3 and 6. The medical system 38 further comprises a medical lead wire 4 accommodated in a through-hole 8 of the lead wire slide 2 or in in a through-hole 8 of the first 2 or the second 2′ (or a third 2″) lead wire slide of the assembly group 34 in a slidable manner. One end of the medical lead wire 4 is connected to a cup or electrode 40 and the other end is connected to (hub) connector 42, and the lead wire slide 2 or the assembly group 34 is provided along the medical lead wire 4 in between the electrode 40 and the connector 42. The lead wire slide 2 or the assembly group 34 is prevented from (unintentionally) falling off or being detached or removed from the medical lead wire 4 by the electrode 40 and the connector 42 (without removing the electrode 40 or connector 42 first, which is generally not contemplated). For assembly purposes the medical lead wire 4 already connected to connector 42 and not connected to the electrode 40 may be guided through the through-hole 8, wherein the electrode 40 is connected to the medical lead wire 4 only afterwards. The medical system 38 is preferably supplied to a customer as a finished goods set comprising the lead wire slide 2 provided on a medical lead wire 4 having the connector 42 at one end and the electrode 40 at the other end. The medical system 38 may comprise a number of medical lead wires 4, such as 5-10 medical lead wires 4, forming a lead wire array. The medical system 38 may be intended for single-patient use.

The following items are examples of various embodiments disclosed above:

1. Lead wire slide (2) for slidable accommodation of medical lead wires (4), comprising: a base section (6) that includes a plurality of through-holes (8) that are each configured to accommodate one medical lead wire (4) in a slidable manner; and a connecting section (10) that is provided on a side (12) of the base section (6) and configured to be connected to a complementary connecting section (10′) of another lead wire slide (2′).

2. Lead wire slide (2) according to item 1, wherein the through-holes (8) are arranged next to each other on a line and run in parallel.

3. Lead wire slide (2) according to item 1 or 2, wherein the lead wire slide (2) is made of polymer material.

4. Lead wire slide (2) according to any of items 1 to 3, wherein the connecting section (10) is configured to form a force-fit connection to a complementary connecting section (10′) of another lead wire slide (2′) and to release the force-fit connection under a force of 4.5 to 10 N.

5. Lead wire slide (2) according to any of items 1 to 4, wherein the lead wire slide (2) includes a further connecting section (14) provided on a side (12) of the base section (6) opposite to the side (12) of the connecting section (10).

6. Lead wire slide (2) according to item 5, wherein the connecting section (10) and the further connecting section (14) are complementary counterparts.

7. Lead wire slide (2) according to item 5 or 6, wherein the connecting section (10) includes a male connecting portion (18) and the further connecting section (14) includes a female connecting portion (16) or vice versa, and wherein the male connecting portion (18) and the female connecting portion (16) are configured to form a force-fit and/or form-fit connection with the respective other one of another lead wire slide (2′).

8. Lead wire slide (2) according to item 7, wherein one of the female connecting portion (16) and the male connecting portion (18) includes a protrusion (22) and the other one includes a complementary recess (24), wherein the protrusion (22) and the recess are (24) configured to snap together with the respective other one of another lead wire slide (2′).

9. Lead wire slide (2) according to any of items 6 to 8, wherein the connecting section (10) includes a male connecting portion (18) and a female connecting portion (16), and the further connecting section (14) includes a female connecting portion (16) complementary to the male connecting portion (18) of the connecting section (10) and a male connecting portion (18) complementary to the female connecting portion (16) of the connecting section (10).

10. Lead wire slide (2) according to any of items 1 to 9, comprising corners and/or edges (26) that are smoothed.

11. Lead wire slide (2) according to any of items 1 to 10, wherein the base section (6) includes at least one groove (32) that is configured to accommodate one medical lead wire (4) in a slidable manner.

12. Assembly group (34) including a first lead wire slide (2) and a second lead wire slide (2′), both according to any of items 1 to 11, wherein the first lead wire slide (2) and the second lead wire slide (2′) are connected or connectable by connecting the connecting section (10) of the first lead wire slide (2) and the connecting section (10′) of the second lead wire slide (2′) that are complementary counterparts and configured to be connected to each other.

13. Assembly group (34) according to item 12, wherein the first lead wire slide (2) and the second lead wire slide (2′) are connected or connectable so that all through-holes (8) of the first lead wire slide (2) and of the second lead wire slide (2′) run in parallel to each other.

14. Medical system (38) comprising a lead wire slide (2) according to any one of items 1 to 11 or an assembly group (34) according to item 12 or 13, and further comprising a medical lead wire (4), wherein the medical lead wire (4) is accommodated in a through-hole (8) of the lead wire slide (2) or in in a through-hole (8) of the first lead wire slide (2) or the second lead wire slide (2′) of the assembly group (34) in a slidable manner.

15. Medical system (38) according to item 14, wherein one end of the medical lead wire (4) is connected to an electrode (40) and the other end is connected to a connector (42), and the lead wire slide (2) or the assembly group (34) is provided along the medical lead wire (4) in between the electrode (40) and the connector (42).

### LIST OF REFERENCE NUMBERS

2 lead wire slide or first lead wire slide
2′ another or further or second lead wire slide
2″ third lead wire slide
4 medical lead wire
6 base section
8 through-hole
10 (first) connecting section
10′ complementary connecting section (of another lead wire slide)
12 side
12a top
12b bottom
12c front
12d back
12e left
12f right
14 further or second connecting section
16 female connecting portion
18 male connecting portion
20 inlet bevel or chamfer
22 protrusion
24 recess
26 edge or corner
28 fillet
30 chamfer
32 groove
34 assembly group
36 gap
38 medical system
40 electrode
42 connector

1. A first lead wire slide comprising:
a base section including through-holes, a first side, and a second side opposite the first side, each of the through-holes being closed-sided and configured to accommodate one medical lead wire in a slidable manner; and
a first connecting section configured to mate with a second connecting section of a second lead wire slide to attach the first lead wire slide to the second lead wire slide.

2. The first lead wire slide of claim 1, wherein the through-holes are arranged next to each other along a straight line and extend in parallel between the first side and the second side of the base section.

3. The first lead wire slide of claim 1, wherein the first lead wire slide is comprised of a polymer material and is devoid of any metal material.

4. The first lead wire slide of claim 1, the first lead wire slide further comprising a second connecting section, wherein the first connecting section extends from the first side and the second connecting section extends from the second side.

5. The first lead wire slide of claim 4, wherein the first connecting section comprises a female connecting portion extending inwardly from the first side of the base section, and wherein the second connecting section comprises a male connecting portion extending outwardly from the second side of the base section.

6. The first lead wire slide of claim 5, wherein the first connecting section also comprises a male connecting portion extending outwardly from the first side of the base section, and wherein the second connecting section also comprises a female connecting portion extending inwardly from the second side of the base section.

7. The first lead wire slide of claim 4, wherein the first connecting section and the second connecting section of the first lead wire slide are complementary counterparts.

8. The first lead wire slide of claim 7, wherein the first connecting section and the second connecting section of the first lead wire slide are configured to form force-fit connections with complementary connecting sections of other lead wire slides and to release each of the force-fit connections under a force of 4.5 to 10 N.

9. The first lead wire slide of claim 8, wherein the first connecting section and the second connecting section each comprises a component of a snap connection, the snap connection including a recess component and a protrusion component, the recess component extending inwardly from a surface of the first connecting section or the second connecting section, and the protrusion component extending outwardly from the other of the first connecting section or the second connecting section.

10. The first lead wire slide of claim 1, further comprising:
a second connecting section configured to mate with a first connecting section of a third lead wire slide to attach the first lead wire slide to the third lead wire slide, and
wherein the first connecting section comprises a female connecting portion extending inwardly from the first side of the base section and the second connecting section comprises a male connecting portion extending outwardly from the second side of the base section.

11. The first lead wire slide of claim 10, wherein the first connecting section also comprises a male connecting portion extending outwardly from the first side of the base section, and wherein the second connecting section also comprises a female connecting portion extending inwardly from the second side of the base section.

12. The first lead wire slide of claim 10, wherein the first connecting section and the second connecting section each comprises a component of a snap connection, the snap connection including a recess component and a protrusion component, the recess component extending inwardly from a surface of the first connecting section or the second connecting section, and the protrusion component extending outwardly from the other of the first connecting section or the second connecting section.

13. The first lead wire slide of claim 12, wherein the second connecting section of the second lead wire slide is identical to the second connecting section of the first lead wire slide, and wherein the first connecting section is configured to form force-fit connection with the second connecting section of the second lead wire slide and to release the force-fit connection under a force of 4.5 to 10 N.

14. The first lead wire slide of claim 1, wherein the second connecting section of the first lead wire slide is configured to mate with a first connecting section of a third lead wire slide to attach the first lead wire slide to the third lead wire slide.

15. The first lead wire slide of claim 14, wherein one of the female connecting portion and the male connecting portion includes a protrusion and the other of the female connecting portion and the male connecting portion includes a complementary recess, wherein the protrusion is configured to form a snap connections with a complementary recess of the second lead wire slide.

16. The first lead wire slide of claim 1, the first lead wire slide further comprising corners and/or edges that are smoothed.

17. The first lead wire slide of claim 1, wherein the base section includes at least one groove configured to accommodate one medical lead wire in a slidable manner.

18. An assembly group including the first lead wire slide of claim 1, the second lead wire slide, and a third lead wire slide, wherein the first lead wire slide, the second lead wire slide, and the third lead wire slide are identical, and wherein the first lead wire slide, the second lead wire slide, and the third lead wire slide are configured to be removably attached to each other.

19. The assembly group of claim 18, wherein all through-holes of the first lead wire slide and of the second lead wire slide run in parallel to each other.

20. An assembly group including the first lead wire slide of claim 1 and the second lead wire slide, wherein the second lead wire slide comprises:
a base section including through-holes, a first side, and a second side opposite the first side, each of the through-holes being configured to accommodate one medical lead wire in a slidable manner;
a first connecting; and
the second connecting section.

21. A medical system comprising:
the first lead wire slide of claim 1; and
a medical lead wire,
wherein the medical lead wire is accommodated in one of the through-holes of the first lead wire slide.

22. The medical system of claim 21, wherein the medical lead wire comprises a first end and a second end, wherein the first end is connected to an electrode and the second end is connected to a connector, and wherein the first lead wire slide is provided along the medical lead wire in between the electrode and the connector.
